(19) United States
(12) Patent Application Publication
(10) Pub. No.: US 2008/0251085 A1
(43) Pub. Date: Oct. 16, 2008

(54) Disposable sleep mask for treating dry eye syndrome

(76) Inventor: Mary Schwebel, Minneapolis, MN (US)

Correspondence Address: PATTERSON, THUENTE, SKAAR & CHRISTENSEN, P.A., 4800 IDS CENTER, 80 SOUTH 8TH STREET, MINNEAPOLIS, MN, 55402-2100, US

(21) Appl. No.: 11/786,156

(22) Filed: Apr. 11, 2007

(51) Int. Cl. A61F 9/04

(52) U.S. Cl. 128/858

(57) ABSTRACT

A mask to ameliorate the effects of dry eye syndrome in a dry eye subject including a thin, flexible, substantially water vapor impermeable membrane having two opposed eye covering portions separated by a central portion. The central portion is gathered to create a shape that accommodates nose structure of the dry eye subject and curves the opposed eye covering portions to conform the opposed eye covering portions generally to the curvature of the dry eye subject's face in an area surrounding the dry eye subject's eyes. The mask further includes an absorbent structure securable between the impermeable membrane and the eyes of the dry eye subject that is capable of absorbing and evaporatively releasing a substance to improve the comfort and health of the eyes and/or adnexa of the dry eye syndrome subject.

### FIELD OF THE INVENTION

The present invention generally relates to masks to be worn over the eyes to provide treatment for dry eye syndrome.

### BACKGROUND OF THE INVENTION

Dry eye syndrome is one of the most commonly treated eye problems in the United States. Dry eye syndrome is also known as keratitis sicca, keratoconjunctivitis sicca (KCS) xerophthalmia, and lacrimal insufficiency. It is estimated that over ten million Americans and 30 million persons worldwide suffer from dry eye syndrome.

For a large fraction of dry eye patients, dry eye syndrome creates discomfort or annoyance. For those severely afflicted, dry eye syndrome can be debilitating and, in some circumstances, even sight-threatening. In extremely severe cases, dry eye syndrome can even lead to the loss of an eye.

Dry eye syndrome typically results from deficiency in the quality or quantity of tears produced by the patient. Precorneal tear film has traditionally been considered to have a three-layered structure. The closest to the cornea lies the mucin, or mucus, layer. The mucin layer provides an interface between the corneal epithelium and the remainder of the tear film. Overlying the mucin layer is the watery aqueous layer, which is the thickest layer of the three. The outermost layer of the precorneal tear film is the lipid layer. The lipid layer is an oily film that reduces evaporation from the aqueous layer beneath it.

The middle aqueous layer provides moisture to the corneal tissue, carries important nutrients, and serves to remove metabolic waste produced by the cornea. Deficiency in any of the three layers of the precorneal tear film can result in complaints of dry, gritty feeling or burning eyes.

The mucin that forms the mucin layer, nearest the cornea, is secreted by goblet cells in the conjunctiva. The conjunctiva is the transparent tissue that covers the sclera and the backside of the eyelids. The mucin layer functions to decrease surface tension of the tear film. In addition, the cornea itself is hydrophobic. Without the mucin layer to provide a bridge between the cornea and the aqueous layer, the aqueous layer would bead up and allow dry spot formation on the cornea.

The aqueous layer is secreted primarily by the glands of Wolfring and Krause located in the eyelid margin. The aqueous layer helps provide an optically smooth, transparent surface to the precorneal tear film. The lipid layer is secreted by the meibomian glands, and the glands of Zeiss and Moll. The glands of Zeiss and Moll are also located at the eyelid margin.

Blinking is essential to maintenance of the precorneal tear film. During each blink, the eyelid wipes over the surface of the cornea, smoothing the mucin layer and spreading the overlying aqueous and lipid layers to provide a completely wetted surface. In between blinks, the tear film thins due to evaporation of the aqueous layer. If evaporation is excessive, dry spots may form on the surface of the cornea.

Deficiency, or imperfect quality, of any of the three component layers can lead to dry eye symptoms. Many systemic and external factors can contribute to dry eye syndrome. For example, Sjogren's syndrome is associated with arthritic diseases in combination with dry eye and dry mouth. Deficiency of Vitamin A, use of oral contraceptives and environmental factors can all contribute to dry eye syndrome.

Recent research into the natural history of dry eye syndrome has shown that the disease progresses through four stages. Each stage is a consequence of the preceding stage. The stages are:

1. Loss of water from the aqueous layer of the tear film leading to an increase in the tear film osmolarity;

2. Loss of conjunctival goblet cells and decreased corneal glycogen;

3. Increased loss of corneal squamous epithelial cells;

4. Destabilization of the interface between the corneal surface and the tear film.

Either decreased secretion of tear film components or increased evaporation lead to increased tear film osmolarity and the following stages that lead to eventual corneal decompensation and the serious consequences of dry eye syndrome.

The adnexa of the eye may also be involved in dry eye syndrome. The adnexa of the eye include the structures surrounding the eye such as the eyelids, eye lashes, the tear drainage and tear production structures. Blepharitis commonly contributes to dry eye syndrome. Blepharitis typically results from bacterial infection of the tiny glands in the margin of the eyelid. These glands include the glands of Zeiss, Moll and Wolfring as well as the meibomian glands. Most commonly, the affected glands are the meibomian glands. In bacterial blepharitis, bacterial infection causes the meibomian glands to become plugged, and thus not be able to produce a normal lipid layer to contribute to the tear film. Some bacteria that infect the glands also secrete exotoxins that seep out of the glands into the eye and injure the corneal epithelium.

Treatments of dry eye syndrome vary depending upon the type of presentation. The most common treatment for dry eye syndrome is the use of artificial tear supplements to provide additional moisture and lubrication to the corneal surface. Artificial tear eye drops are placed on the eye by the patient. Artificial tear supplements must be used regularly and often to be effective.

Lubricant ointments may also be employed. Ointments are usually used at bedtime because they tend to be messy and blur vision. For some patients, even the use of ointments is not sufficient to provide comfort during sleep.

Tears drain from the eye through the lacrimal drainage system. Tiny openings at the nasal corner of each upper and lower eyelid are called the lacrimal puncta. The lacrimal puncta lead into ducts that drain into the nasopharynx

One treatment for dry eye syndrome is to partially or completely close one or more lacrimal puncta to reduce tear outflow into the lacrimal drainage apparatus. Traditionally, this closure was accomplished surgically or by cautery. In the last decade, however, temporary and permanent punctal occlusion plugs have been utilized.

Permanent punctal plugs are typically made from surgical silicone; temporary plugs are generally made of soluble collagen. Collagen plugs dissolve over a period of days and are helpful in diagnosis.

Punctal plugs are placed into the lacrimal puncta, or lacrimal drainage ducts. The plugs impede the outflow of tears from the eye. This approach slows the outflow of tears and retains them in the eyes longer, often relieving symptoms. Punctal plugs have the distinct advantage of being readily removable and avoid the issues of scar formation.

Blepharitis is sometimes treated by the use of antibiotic medications. Another important treatment for blepharitis is the application of warm soaks and lid scrubs. In this form of treatment, the patient applies a warm wet washcloth to the eyelids for a period of time to provide humidity, warmth and to help soften blockage of and restore flow from the meibomian glands. Lid scrubs are practiced by taking a mild, nonirritating soap and vigorously scrubbing the eyelid margins with they eyes closed, so as to massage the meibomian glands and increase production. The surfactant helps to dissolve the greasy blockage of the meibomian glands.

Patients who have severe dry eye syndrome often suffer disrupted sleep because they cannot go for longer than an hour or so without applying tear supplements to the eyes. This can lead to pronounced sleep deprivation and a consequent reduction in quality of life.

A variety of researchers have been seeking other medicinal treatments for dry eye syndrome. Largely, this research is directed at pharmaceutical efforts to increase tear production. In addition it has been discovered that an inflammatory component plays a larger part in dry eye syndrome than was previously thought. Recent research has led to some success in treating the inflammatory component of dry eye syndrome. Restasis® is a topical ophthalmic solution containing the anti-rejection drug Cyclosporin.

Despite the many treatment options available, there remains no cure for dry eye syndrome. A great many patients still have substantial and even debilitating discomfort because of dry eye syndrome. Very few treatment options exist to provide comfort for dry eye syndrome patients during sleep.

Sleep masks to treat dry eye syndrome can provide a higher humidity environment in the vicinity of the eyes and adnexa to improve the comfort and health of the eyes. Such masks are disclosed in U.S. Pat. Nos. 7,036,928, 6,886,933, 6,874,884 and 6,641,264 issued to and commonly owned by the inventor named in this application. In addition, U.S. published applications 2006/0157064 and 2005/0022823 also disclose sleep masks to treat dry eye syndrome. Existing masks on the market to ameliorate the effects of dry eye syndrome generally are expensive and relatively complex structures. Dry eye sufferers would benefit from a simple light and comfortable sleep mask. Additionally, dry eye patients would benefit from a sleep mask that is inexpensive and disposable in nature.

### SUMMARY OF THE INVENTION

The present invention solves many of the above discussed problems. The present invention includes a mask to ameliorate the effects of dry eye syndrome. The mask includes a thin, flexible water vapor impermeable membrane structured to cover on or two eyes. The mask may be formed from a substantially planar sheet of thin flexible polymer material.

The thin polymer material is formed to conform to the shape of the face and eyes by the use of gathering or elastic materials. The mask, in one of the aspect of the invention, includes two opposed eye covering portions that are separated by a central portion. The central portion is gathered to create a shape that accommodates the nose of the dry eye subject using the mask. In addition, the gathering in the nasal portion of the mask curves the opposed eye covering portions to comfortably conform them to the curvature of the patient's face surrounding the eyes.

In another aspect of the invention, the mask includes an absorbent structure, which is secured or securable to the back side of the thin polymer membrane. The absorbent structure is capable of absorbing and evaporatively releasing a fluid substance to improve the comfort and/or health of the eyes and surrounding structures of the dry eye syndrome subject.

The mask of the present invention further includes a retaining member to secure the mask to the head of the dry eye syndrome sufferer. The retaining member may include an elastic strap that can be extended around the subject's head. In another embodiment of the invention, the retaining member may include adhesive tabs that can be secured to the skin of the dry eye subject in the area of the temples. Any other retaining member known to those of skill in the art can also be used to secure the mask of the present invention.

The thin, flexible membrane of the present invention may be formed from a thin polymer film sheet. For example, the thin, flexible membrane may be formed from a sheet of polyethylene plastic, having a thickness ranging from about 20 mils to about 5 mils. The thin, flexible membrane of the present invention may be made from any appropriate water vapor impermeable polymer substance or another material, such as thin metal foil or mylar.

The formed portions or gathered central portion of the thin membrane may be formed by a variety of methods. The gathered central portion may be formed, for example, by a stitching procedure similar to that used to gather fabric. The gathered portion may also be formed by the use of adhesives or heat techniques to fuse the thin membrane material so that it retains the gathered structure centrally. Elastic members may be used to shape and form the mask as desired to conform to a desired shape to cover desired portions of the face in the vicinity of the eyes.

### BRIEF DESCRIPTION OF THE DRAWINGS

FIG. 1 is a perspective view of a sleep mask in accordance with the present invention;

FIG. 2 is an elevational view of the sleep mask;

FIG. 3 is cross-sectional view of the sleep mask in accordance with the present invention;

FIG. 4 is a perspective view of a sleep mask including an elastic retaining member as used on the pad of a dry eye subject in accordance with the present invention; and

FIG. 5 is a perspective view of a sleep mask in accordance with the present invention including an adhesive retaining member on the head of a dry eye subject in accordance with the present invention.

### DETAILED DESCRIPTION

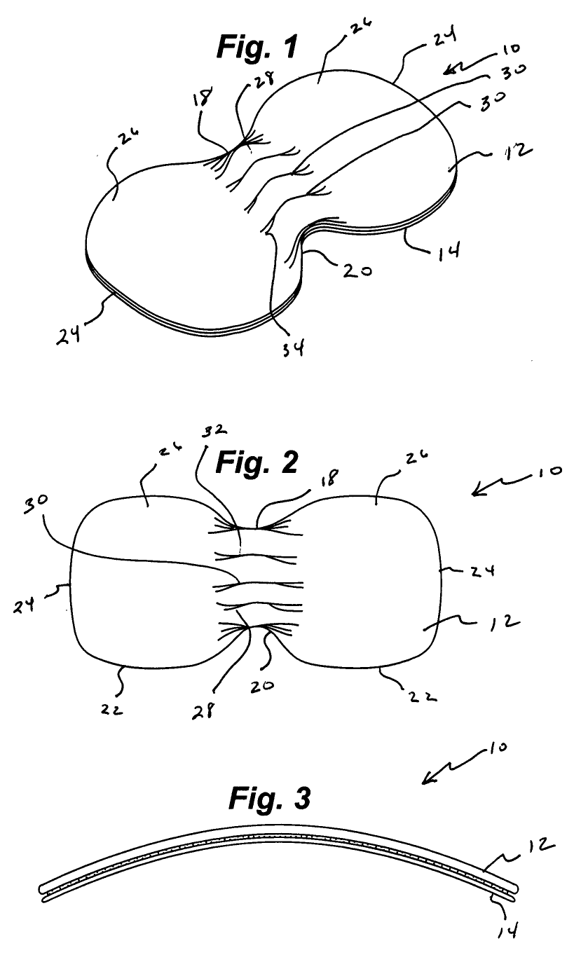

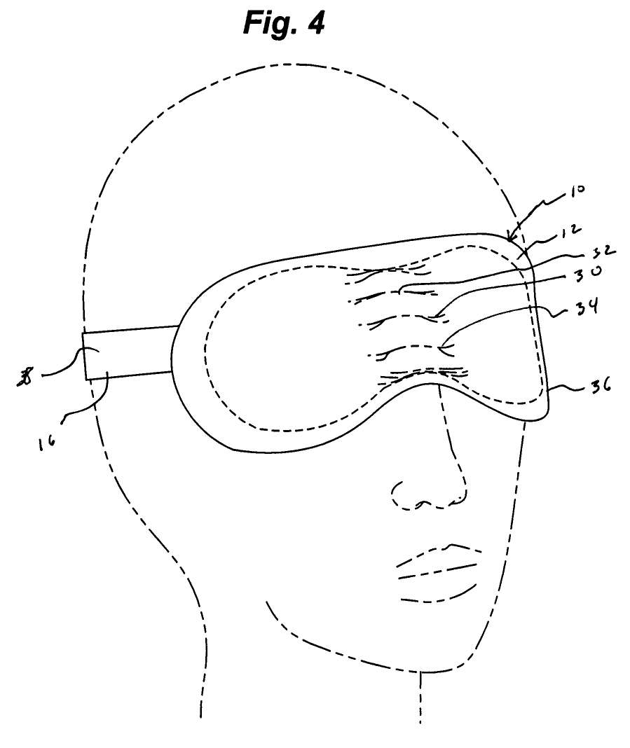

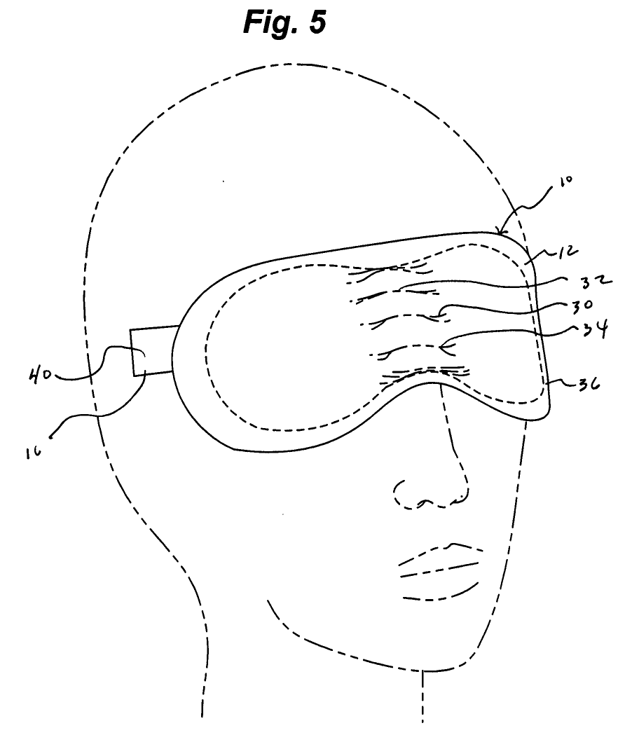

Referring to FIGS. 1-3, eye mask 10 generally includes membrane 12 and absorbent member 14. Referring to FIGS. 4 and 5, eye mask 10 may optionally include retainer 16. Referring to FIGS. 1 and 2, membrane 12 may include or define brow arch 18, nose relief 20, cheek arches 22, temple curves 24, eye portions 26 and central portion 28. Eye portions 26 flank central portion 28. Central portion 28 is bounded above by brow arch 18 and below by nose relief 20. Eye portions are bounded by cheek arches 22 and temple curves 24.

Membrane 12 may define gathers 30, folds 32 or include elastic members 34.

Gathers 30 or folds 32 may be located centrally as depicted in FIGS. 1, 2, 4 and 5. The effect of gathers 30 on central portion 28 is to create a curved shape that accommodates the nose of the dry eye subject using the mask. Further, gathers 34 tend to curve eye portions 26 so that they conform to the curvature of the patient's face surrounding the eyes. Elastic members 34 may be incorporated generally vertically in central portion 28 to assist in the forming of gathers 30 or folds 32. Elastic members 34 may also be utilized around a perimeter 36 of eye portions 26 in order to assist in curving and forming membrane 12 and absorbent member 14.

Absorbent member 14 is secured to membrane 12. Absorbent member 14 may be secured to membrane 12 around perimeter 36 or over the surfaces of absorbent member 14 and membrane 12. Absorbent member 14 may include super absorbent particles. Absorbent member 14 may be a single structure that covers most of membrane 12 or may include two or more portions that cover parts of membrane 12.

Referring to FIGS. 4 and 5, retainer 16 may include an elastic member 38 or adhesive members 40. Elastic member 38 as depicted in FIG. 4 may extend around the head of a user of the eye mask 10. Adhesive members 40 may be adhered to the skin of a user to secure eye mask 10 in position. Retainer 16 may also embody any other type of retaining member known to those of skill in the art. Membrane 12 may be formed of a thin flexible polymers film sheet or another water vapor impermeable substance such as thin metal foil or Mylar. In one aspect of the invention, membrane 12 may be formed from a sheet of polyethylene plastic having a thickness ranging from about 20 mils to about 5 mils. Membrane 12 may be formed from any appropriate water vapor impermeable material.

Gathers 30 and folds 32 may be formed by a variety of known methods. Gathers 30 may be formed for example by stitching in a procedure similar to that used to gather fabric. Gathers 30 or folds 32 may also be formed by the use of adhesives or heat techniques or by the insertion of elastic members 34.

The present invention may be embodied in other specific forms without departing from the spirit of any of the essential attributes thereof; therefore, the illustrated embodiment should be considered in all respects as illustrative and not restrictive, reference being made to the appended claims rather than to the forgoing description to indicate the scope of the invention.

What is claimed is:

1. A mask to ameliorate the effects of dry eye syndrome in a dry eye subject, the mask comprising:
a thin, flexible, substantially water vapor impermeable membrane having two opposed eye covering portions separated by a central portion, in which the central portion is gathered to create a shape that accommodates nose structure of the dry eye subject and that curves the opposed eye covering portions to conform the opposed eye covering dry eye subject's eyes;
an absorbent structure securable between the impermeable membrane and the eyes of the dry eye subject, the absorbent structure being capable of absorbing and evaporatively releasing a substance to improve the comfort and health of the eyes and/or adnexa of the dry eye syndrome subject; and
a retaining member to secure the mask to the head of the dry eye subject.

2. The mask as claimed in claim 1, in which the retaining member is adapted to at least partially encircle the head of the dry eye subject.

3. The mask as claimed in claim 1, in which the retaining member comprises an adhesive member whereby the mask can be secured to the dry eye subject's skin.

4. The mask as claimed in claim 1, in which the vapor impermeable membrane is formed of a polymer and has a thickness less than about 20 mils.

5. The mask as claimed in claim 1, in which the vapor impermeable membrane is formed of a polymer and has a thickness less than about 10 mils.

6. The mask as claimed in claim 1, in which the vapor impermeable membrane is formed of a polymer and has a thickness less than about 5 mils.

7. The mask as claimed in claim 1, in which the gathered portion includes an elastic member.

8. The mask as claimed in claim 1, in which the gathered portion comprises an adhesive.

9. The mask as claimed in claim 1, in which the gathered portion comprises heat formed structure.

10. A method of making a mask to ameliorate the affects of dry eye syndrome, comprising:
cutting a portion of a thin flexible water vapor impermeable membrane to a desired shape to cover eyes of a dry eye syndrome subject;
gathering a central portion of the thin flexible water vapor impermeable membrane to cause the thin flexible water vapor impermeable to curve to conform to a shape that accommodates a user's facial structure in a vicinity of the eyes and nose;
securing an absorbent structure to the thin flexible water vapor impermeable membrane;
securing a retaining member to the thin flexible water vapor impermeable membrane or to the absorbent structure.

11. The method as claimed in claim 10, further comprising structuring the retaining member to at least partially encircle the head of the dry eye subject.

12. The method as claimed in claim 10, further comprising structuring the retaining member to include an adhesive member to secure the mask to the dry eye subject's skin.

13. The method as claimed in claim 10, further comprising forming the vapor impermeable membrane of a polymer that has a thickness less than about 20 mils.

14. The method as claimed in claim 10, further comprising forming the vapor impermeable membrane of a polymer that has a thickness less than about 10 mils.

15. The method as claimed in claim 10, further comprising forming the vapor impermeable membrane of a polymer that has a thickness less than about 5 mils.

16. The method as claimed in claim 10, further comprising incorporating an elastic member into the gathered portion.

17. The method as claimed in claim 10, further comprising forming the gathered portion with an adhesive.

18. The method as claimed in claim 10, further comprising forming the gathered portion by the application of heat to the membrane.
